(19) United States
(12) Patent Application Publication
(10) Pub. No.: US 2023/0404913 A1
(43) Pub. Date: Dec. 21, 2023

(54) PHARMACEUTICAL FORMULATIONS FOR TREATING DISEASES ASSOCIATED WITH VOLTAGE-GATED SODIUM CHANNELS

(71) Applicant: Xenon Pharmaceuticals, Inc., Burnaby (CA)

(72) Inventors: Jean-Jacques Alexandre Cadieux, Burnaby (CA); Matthew David Tandy, Vancouver (CA)

(21) Appl. No.: 18/248,661

(22) PCT Filed: Apr. 9, 2021
(86) PCT No.: PCT/CA2021/050470

§ 371 Date: Apr. 11, 2023

Related U.S. Application Data
(60) Provisional application No. 63/090,860, filed on Oct. 13, 2020.
(60) Provisional application No. 63/090,872, filed on Oct. 13, 2020.
(60) Provisional application No. 63/090,875, filed on Oct. 13, 2020.

(51) Int. Cl. A61K 9/00; A61K 9/16; A61K 9/20; A61K 9/10; A61K 9/28; A61K 31/635; A23L 33/00; A23L 33/125

(52) U.S. Cl. CPC A61K 9/0053; A61K 9/1623; A61K 9/1652; A61K 9/1617; A61K 9/2054; A61K 9/2013; A61K 9/10; A61K 9/2866; A61K 31/635; A23L 33/40; A23L 33/125

(57) ABSTRACT

Provided herein are certain pharmaceutical compositions comprising (S)-4-((1-benzylpyrrolidin-3-yl)(methyl)amino)-2-fluoro-5-methyl-N-(thiazol-4-yl)benzene-sulfonamide (Compound A), or a pharmaceutically acceptable salt thereof, and at least one pharmaceutically acceptable excipient. The pharmaceutical compositions can be present in form of an immediate release tablet, a granular formulation, a stable suspension oral dosage form or a multi-particulate sprinkle dosage form. Methods for the treatment of a disease or a condition associated with Naj4.6 activity, such as epilepsy, are described.

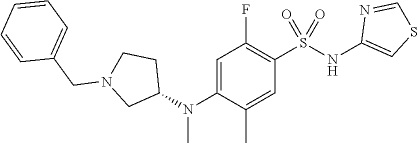 [SMILES: Cc1cc(S(=O)(=O)Nc2cscn2)c(F)cc1N(C)[C@H]1CCN(Cc2ccccc2)C1]

This application claims priority to, and the benefit of, U.S. Application Nos. 63/090,860, 63/090,872, and 63/090,875, each of which was filed on Oct. 13, 2021, the entirety of each is incorporated by reference herein.

Voltage gated sodium channels (Na_{V}'s) are critical determinants of cellular excitability in muscle and nerve. Four isoforms in particular, Na_{V}1.1, Na_{V}1.2, Na_{V}1.3, and Na_{V}1.6, account for the majority of sodium current in the neurons of the central nervous system. Na_{V}1.3 is primarily expressed embryonically. Beyond the neonatal stage, Na_{V}1.1, Na_{V}1.2, and Na_{V}1.6 are the critical isoforms that regulate neuronal signaling in the brain.

Epilepsy is a condition characterized by excessive synchronous excitability in the brain that arises when the delicate balance of excitatory and inhibitory signals in the brain fall out of equilibrium. This can happen either due to an excess of excitation, or a deficiency of inhibition. Mutations in the genes encoding Na_{V} channels have been linked to both types of disequilibrium.

Voltage-gated sodium channel antagonism is the most common mechanism of widely prescribed antiepileptic drugs (AED's). Carbamazepine, eslicarbazepine, oxcarbazepine, lacosamide, lamotrigine, phenytoin, rufinamide and zonisamide are all believed to act primarily by blocking that function of Na_{V} channels. Despite the presumed mechanism of action, these drugs are relatively promiscuous. They block all Na_{V} channel isoforms indiscriminately, thus block of Na_{V}1.1 would be expected to proconvulsant. Block of Na_{V}1.6, and perhaps Na_{V}1.2, would be anticonvulsant. In addition to sodium channels, these compounds also block other targets, including voltage-gated calcium channels. Selective Na_{V} antagonists that spare Na_{V}1.1 and other off-target receptors are expected to have both improved efficacy and therapeutic index relative to the currently available Na_{V} blocking drugs.

Thus, there is a need in the art for new orally administered treatments for epilepsy and other Na_{V}1.6 associated pathological states. The present invention provides treatments to meet these critical needs.

### SUMMARY

Provided is a pharmaceutical formulation comprising:
S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof and
at least one pharmaceutically acceptable carrier.

Provided is an immediate release tablet comprising:
(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
at least one pharmaceutically acceptable diluent;
at least one pharmaceutically acceptable disintegrant;
at least one pharmaceutically acceptable glidant;
at least one pharmaceutically acceptable wetting agent; and
at least one pharmaceutically acceptable lubricant.

Also provided is a method of treating a disease or a condition associated with Na_{V}1.6 activity in a patient in need thereof, the method comprising: administering to the patient in need thereof a therapeutically effective amount of an immediate release tablet as described herein.

Also provided is a granular formulation suitable for reconstitution with a pharmaceutically acceptable carrier to form a stable suspension oral dosage form comprising one or more pharmaceutically acceptable excepients and an amount of (S)-4-((1-benzylpyrrolidin-3-yl)(methyl)amino)-2-fluoro-5-methyl-N-(thiazol-4-yl)benzenesulfonamide (Compound A) or a pharmaceutically acceptable salt thereof.

Also provided is a granular formulation suitable for reconstitution with a pharmaceutically acceptable carrier to form a stable suspension oral dosage form comprising:
(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
at least one pharmaceutically acceptable filler or diluent;
at least one pharmaceutically acceptable binder; and
at least one pharmaceutically acceptable lubricant.

Also provided is a stable suspension oral dosage form comprising the granular formulation as described herein that has been reconstituted with a pharmaceutically acceptable carrier.

Also provided is a method of administering Compound A, or a pharmaceutically acceptable salt thereof, to a patient in need thereof, comprising:
(a) providing a granular formulation as described herein;
(b) reconstituting the granular formulation to produce a stable suspension oral dosage form; and
(c) administering the stable suspension oral dosage form.

Also provided is a method of administering Compound A, or a pharmaceutically acceptable salt thereof, to a patient in need thereof, comprising: orally administering the stable suspension oral dosage form as described herein.

Also provided is a method of treating a disease or a condition associated with Na_{V}1.6 activity, the method comprising administering the stable suspension oral dosage form as described herein to a patient in need thereof.

Provided is a multiparticulate sprinkle dosage form comprising a plurality of discrete units, wherein each unit comprises one or more pharmaceutically acceptable excipients and an amount of (S)-4-((1-benzylpyrrolidin-3-yl)(methyl)amino)-2-fluoro-5-methyl-N-(thiazol-4-yl)benzenesulfonamide (Compound A) or a pharmaceutically acceptable salt thereof.

Also provided is a multiparticulate sprinkle dosage form comprising a plurality of discrete units, wherein each unit comprises:
(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
at least one pharmaceutically acceptable filler,
at least one pharmaceutically acceptable binder, and
at least one pharmaceutically acceptable lubricant.

Also provided is a method of administering Compound A, or a pharmaceutically acceptable salt thereof, to a patient in need thereof, comprising:
(a) providing a multiparticulate sprinkle dosage form described herein;
(b) sprinkling the discrete units on soft food; and
(c) administering the soft food orally.

Also provided is a method of administering Compound A, or a pharmaceutically acceptable salt thereof, to a patient in need thereof, comprising: orally administering a multiparticulate sprinkle dosage form described herein.

Also provided is a method of administering Compound A, or a pharmaceutically acceptable salt thereof, to a patient in need thereof, comprising: orally administering a capsule containing the multiparticulate sprinkle dosage form described herein.

Also provided is a method of treating a disease or a condition associated with Na_{V}1.6 activity, the method comprising administering the multiparticulate sprinkle dosage form described herein to a patient in need thereof.

These and other objects of the invention are described in the following paragraphs. These objects should not be deemed to narrow the scope of the invention.

### BRIEF DESCRIPTION OF THE DRAWINGS

FIG. 1 depicts a manufacturing process for a granular formulation suitable for reconstitution as described herein.

FIG. 2 depicts a manufacturing process for an immediate release tablet formulation as described herein. The ingredient numbers are as follows: 1: Compound A; 2: microcrystalline cellulose (MCC); 3: croscarmellose sodium; 4: colloidal silicon dioxide; 5: sodium lauryl sulfate; 6: magnesium stearate; 7: Opadry II (white); and 8: water for injection.

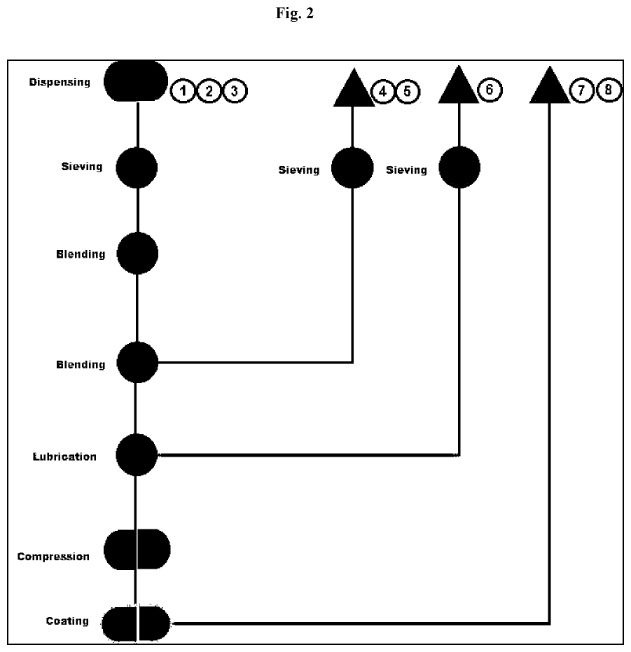

### DETAILED DESCRIPTION OF THE INVENTION

This detailed description is intended only to acquaint others skilled in the art with the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

### A. DEFINITIONS

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” or “some embodiments” or “a certain embodiment” means that a particular feature, structure or characteristic described in connection with the embodiment is included in at least one embodiment. Thus, the appearances of the phrases “in one embodiment” or “in an embodiment” or “in some embodiments” or “in a certain embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise.

As used in the specification and the appended claims, unless specified to the contrary, the following terms have the meaning indicated:

The term “API” as used herein stands for “active pharmaceutical ingredient.” The API as disclosed herein is (S)-4-((1-benzylpyrrolidin-3-yl)(methyl)amino)-2-fluoro-5-methyl-N-(thiazol-4-yl)benzenesulfonamide (Compound A) or a pharmaceutically acceptable salt thereof. Compound A may also be referred to as XEN901 or NBI 921352.

As used herein, the term “pharmaceutical composition” or “pharmaceutical formulation” means a composition comprising Compound A or a pharmaceutically acceptable salt thereof and, optionally, one or more pharmaceutically acceptable excipients.

The term “pharmaceutically acceptable” refers to a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree.

The term “patient” or “individual” or “subject” means a mammal, including a human, for whom or which therapy is desired, and generally refers to the recipient of the therapy.

The term “effective amount” and “therapeutically effective amount” of an agent, compound, drug, composition or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician.

The terms “treating” or “treatment” refers to therapeutic applications to slow or stop progression of a disorder, prophylactic application to prevent development of a disorder, and/or reversal of a disorder. Reversal of a disorder differs from a therapeutic application which slows or stops a disorder in that with a method of reversing, not only is progression of a disorder completely stopped, cellular behavior is moved to some degree, toward a normal state that would be observed in the absence of the disorder.

The term “disorder” is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

The term “administering to a patient” refers to the process of introducing a composition or dosage form into the patient via an art-recognized means of introduction

The term “Cₘₐₓ” refers to the peak concentration and, in particular, the maximum observed plasma/serum concentration of drug.

The term “tₘₐₓ” is a pharmacokinetic parameter denoting the time to maximum blood plasma concentration following delivery of an active pharmaceutical ingredient.

The term “t_{1/2}” or “plasma half-life” or “elimination half-life” or the like is a pharmacokinetic parameter denoting the apparent plasma terminal phase half-life, i.e., the time, after absorption and distribution of a drug is complete, for the plasma concentration to fall by half.

The term “AUC” refers to the area under the curve, or the integral, of the plasma concentration of an active pharmaceutical ingredient or metabolite over time following a dosing event.

The term “AUC₀₋ₜ” is the integral under the plasma concentration curve from time 0 (dosing) to time “t”.

The term “AUC_{0-∞}” is the AUC from time 0 (dosing) to time infinity. Unless otherwise stated, AUC refers to AUC_{0-∞}.

The term “adjusting administration”, “altering administration”, “adjusting dosing”, or “altering dosing” are all equivalent and mean tapering off, reducing or increasing the dose of the substance, ceasing to administer the substance to the patient, or substituting a different active agent for the substance.

The term ““immediate release” pharmaceutical formulation includes any formulation in which the rate of release of drug from the formulation and/or the absorption of drug, is neither appreciably, nor intentionally, retarded by galenic manipulations. Thus, the term excludes formulations which are adapted to provide for “modified”, “controlled”, “sustained”, “prolonged”, “extended” or “delayed” release of drug. In this context, the term “release” includes the provision (or presentation) of drug from the formulation to the gastrointestinal tract, to body tissues and/or into systemic circulation.

The term “stable” refers to both chemical (shelf-life) and physical stability (suspension uniformity). Improved uniformity results in an improved product because less shaking of the suspension is required before dosing and allows the product to be stored longer (i.e. longer shelf-life) because the drug in the product will not settle and compact.

The term “suspension” refers to a mixture of a solid in liquid.

The term “multiparticulate sprinkle dosage form” or “formulation” or “composition”, as used herein, refer to plurality of physically discrete units, wherein each unit contains a predetermined quantity of active ingredient in association with pharmaceutically acceptable excipients.

The term “multiparticulate” as used herein refers to plurality of physically discrete units. The discrete unit comprises a drug core in the form of pellets, beads, particles, granules, or minitablets. In the present specification, the term “multiparticulates”, “discrete units”, “pellets”, “beads”, are used interchangeably.

As used herein, “about” means ±20% of the stated value, and includes more specifically values of ±10%, ±5%, ±2% and ±1% of the stated value.

### B. DRUG SUBSTANCE

Pharmaceutical compositions disclosed herein comprise at least one active pharmaceutical ingredient: (S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof.

Compound A has the following formula:

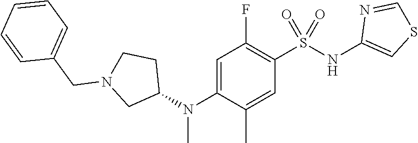 [SMILES: Cc1cc(S(=O)(=O)Nc2cscn2)c(F)cc1N(C)[C@H]1CCN(Cc2ccccc2)C1]

Methods of making Compound A and a pharmaceutically acceptable salt thereof are described in U.S. Pat. No. 10,662,184, the contents of which are herein incorporated by reference.

Compound A may be present in a pharmaceutical composition in the form of acid addition salts. Acid addition salts of the free amino compounds may be prepared by methods well known in the art, and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, p-toluenesulfonic acid, and benzenesulfonic acids. Suitable inorganic acids include hydrochloric, hydrobromic, sulfuric, phosphoric, and nitric acids. Thus, the term “pharmaceutically acceptable salt” of Compound A is intended to encompass any and all acceptable salt forms.

As used herein, and in the absence of a specific reference to a particular pharmaceutically acceptable salt of Compound A, any dosages, whether expressed in milligrams or as a percentage by weight or as a ratio with another ingredient, should be taken as referring to the amount of Compound A. For example, a reference to “20 mg Compound A or a pharmaceutically acceptable salt thereof” means an amount of Compound A or a pharmaceutically acceptable salt thereof that provides the same amount of Compound A as 20 mg of Compound A free form.

In some embodiments, Compound A, or a pharmaceutically acceptable salt thereof, is Compound A free base.

In some embodiments, Compound A, or a pharmaceutically acceptable salt thereof, is a pharmaceutically acceptable salt of Compound A.

In some embodiments, the amount of Compound A, or pharmaceutically acceptable salt thereof, is from about 2 mg to about 100 mg. In some embodiments, the amount of Compound A is about 2.5, about 5, about 10, about 20, about 30, about 40, or about 50 mg. In some embodiments, the amount of Compound A is about 2.5, about 5, about 10, or about 20 mg. In some embodiments, the amount of Compound A is about 2.5 mg. In some embodiments, the amount of Compound A is about 5 mg. In some embodiments, the amount of Compound A is about 10 mg. In some embodiments, the amount of Compound A is about 20 mg. In some embodiments, the amount of Compound A is about 30 mg. In some embodiments, the amount of Compound A is about 40 mg. In some embodiments, the amount of Compound A is about 50 mg. In some embodiments, the amount of Compound A is about 60 mg. In some embodiments, the amount of Compound A is about 70 mg. In some embodiments, the amount of Compound A is about 80 mg. In some embodiments, the amount of Compound A is about 90 mg. In some embodiments, the amount of Compound A is about 100 mg.

In some embodiments, Compound A, or the pharmaceutically acceptable salt thereof, is present in an amount of between about 5 and about 15% w/w, measured as the free base. In some embodiments, Compound A, or the pharmaceutically acceptable salt thereof, is present in an amount of about 10% w/w, measured as the free base.

### C. PHARMACEUTICAL COMPOSITIONS

This disclosure is directed to providing Compound A or a pharmaceutically acceptable salt thereof in a pharmaceutical composition that is pharmacologically efficacious and physically acceptable. The pharmaceutical compositions disclosed herein are intended for pharmaceutical use in human subjects.

1. Immediate Release Tablet

Provided is an immediate release tablet comprising:
(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
at least one pharmaceutically acceptable diluent,
at least one pharmaceutically acceptable disintegrant,
at least one pharmaceutically acceptable glidant,
at least one pharmaceutically acceptable wetting agent, and
at least one pharmaceutically acceptable lubricant.

The disclosure is also directed to an immediate release tablet for use in method of treating a disease or a condition associated with Na_{V}1.6 activity. The disclosure is also directed to an immediate release tablet for use in method of treating epilepsy.

In some embodiments, the at least one pharmaceutically acceptable diluent is chosen from polyols, such as dextrose, xylitol, lactitol, isomalt, mannitol such as spray dried mannitol (e.g., Pearlitol® 100SD, Pearlitol® 200SD), sorbitol, lactose, and sucrose; starches, such as pregelatinized starch, potato or corn starch; cellulose derivatives, such as powdered cellulose, microcrystalline cellulose (e.g., Avicel®) or silicified microcrystalline cellulose; talc; magnesium carbonate, calcium carbonate, calcium dihydrogen phosphate dihydrate, calcium phosphate-dibasic, calcium phosphate-tribasic, or calcium sulfate; dextrates; kaolin; silicic acid; mixtures of isomaltulose derivatives such as galenIQ™ 720; and combinations thereof. In some embodiments, the pharmaceutically acceptable diluent is chosen from mannitol, lactose, starches, dextrose, sorbitol, microcrystalline cellulose, dibasic calcium phosphate dehydrate, sucrose, maltitol, and combinations thereof. In some embodiments, the at least one pharmaceutically acceptable tablet diluent is microcrystalline cellulose.

In some embodiments, the at least one pharmaceutically acceptable tablet diluent is present an amount from about 35% to about 55% by weight (w/w). In some embodiments, the at least one pharmaceutically acceptable tablet diluent is present in an amount from about 40% to about 50% by weight (w/w).

In some embodiments, the at least one pharmaceutically acceptable disintegrant is chosen from starch, pregelatinized starch, croscarmellose sodium, crospovidone, sodium starch glycolate, and combinations thereof. In some embodiments, the at least one pharmaceutically acceptable disintegrant is croscarmellose sodium.

In some embodiments, the at least one pharmaceutically acceptable disintegrant is present in an amount of between about 2 and about 10% w/w. In some embodiments, the at least one pharmaceutically acceptable disintegrant is present in an amount of between about 2.5 and about 3% w/w.

In some embodiments, the at least one pharmaceutically acceptable glidant is chosen from calcium phosphate dibasic, calcium silicate, colloidal silicon dioxide, magnesium silicate, magnesium trisilicate, silicon dioxide, starch, talc, and combinations thereof. In some embodiments, the at least one pharmaceutically acceptable glidant is colloidal silicon dioxide.

In some embodiments, the at least one pharmaceutically acceptable glidant is present in an amount of between about 0.1 and about 5% w/w. In some embodiments, the at least one pharmaceutically acceptable glidant is present in an amount of between about 0.1 and about 1% w/w.

In some embodiments, the at least one pharmaceutically acceptable wetting agent is chosen from polyoxyethyelene alkyl ethers, polyoxyethylene stearates, poloxamers, sodium lauryl sulfate, docusate sodium (dioctyl sulfosuccinate sodium salt), benzalkonium chloride, benzethonium chloride, and cetrimide (alkyltrimethylammonium bromide), polysorbate, sorbitan esters, glyceryl monooleate, and combinations thereof. In some embodiments, ein the at least one pharmaceutically acceptable wetting agent is sodium lauryl sulfate.

In some embodiments, the at least one pharmaceutically acceptable wetting agent is present in an amount of between about 0.1 and about 5% w/w. In some embodiments, the at least one pharmaceutically acceptable wetting agent is present in an amount of between about 0.1 and about 1% w/w.

In some embodiments, the at least one pharmaceutically acceptable lubricant is chosen from colloidal anhydrous silica, croscarmellose sodium, stearic acid, magnesium stearate, glyceryl behenate, calcium stearate, zinc stearate, sodium stearyl fumarate, talc, microcrystalline wax, yellow beeswax, white beeswax, corn starch, polyethylene glycol, surfactants, sodium benzoate, hydrogenated vegetable oil, hydrogenated castor oil, mineral oil, polyoxyethylene stearates, lauryl sulphate salts, glyceryl behenate, glyceryl palmitostearate, and combinations thereof. In some embodiments, the at least one pharmaceutically acceptable lubricant is magnesium stearate.

In some embodiments, the at least one pharmaceutically acceptable lubricant is present in the pharmaceutical composition in an amount from about 0.5% to about 5% by weight (w/w) of the pharmaceutical composition. In some embodiments, the at least one pharmaceutically acceptable lubricant is present in an amount from about 0.5% to about 1% by weight (w/w).

In some embodiments, the immediate release tablet has a film coat. In some embodiments, the film coating is an aqueous film coating. In some embodiments, the film coating comprises Opadry® II White, which is available from Colorcon, Inc., Harleysville, PA and contains polyvinyl alcohol, titanium dioxide, macrogol 3350, and talc. In some embodiments, the film coating comprises Opadry II Blue which comprises polyvinyl alcohol, macrogol 3350, titanium dioxide, talc, indigo carmine aluminium lake. In some embodiments, the film coating comprises Opadry II Yellow which comprises polyvinyl alcohol, macrogol 3350, titanium dioxide, talc, iron oxide yellow. In some embodiments, the film coating comprises Opadry II Orange: polyvinyl alcohol, macrogol 3350, titanium dioxide, talc, sunset yellow FCF aluminium lake, iron oxide red.

In some embodiments, the immediate release tablet has the following composition.

| | Strength (label claim) | |
|---|---|---|
| | 50 mg | |
| | Quantity per unit | |
| Component | (mg) | % |
| Compound A | 50.00 | 46.95 |
| Microcrystalline cellulose | 45.50 | 42.72 |
| Croscarmellose Sodium, Primellose | 3.00 | 2.82 |
| Colloidal Silicon Dioxide, Aerosil 200 | 0.50 | 0.47 |
| Sodium Lauryl Sulfate, Kolliphor SLS | 0.50 | 0.47 |
| Magnesium Stearate | 0.50 | 0.47 |
| Opadry II (white) | 6.50 | 6.10 |
| Total | 106.50 | 100 |

2. Granular Formulation Suitable for Reconstitution and Stable Suspension Oral Dosage Form

Provided is a granular formulation suitable for reconstitution with a pharmaceutically acceptable carrier to form a stable suspension oral dosage form comprising one or more pharmaceutically acceptable excipients and an amount of (S)-4-((1-benzylpyrrolidin-3-yl)(methyl)amino)-2-fluoro-5-methyl-N-(thiazol-4-yl)benzenesulfonamide (Compound A) or a pharmaceutically acceptable salt thereof.

Also provided is a granular formulation suitable for reconstitution with a pharmaceutically acceptable carrier to form a stable suspension oral dosage form comprising:
(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof;
at least one pharmaceutically acceptable filler or diluent;
at least one pharmaceutically acceptable binder; and
at least one pharmaceutically acceptable lubricant.

The disclosure is also directed to a granular formulation suitable for reconstitution for use in method of treating a disease or a condition associated with Na_{V}1.6 activity. The disclosure is also directed to a granular formulation suitable for reconstitution for use in method of treating epilepsy.

In some embodiments, the granular formulation suitable for reconstitution comprises at least one pharmaceutically acceptable excipient that functions as a filler or diluent. Fillers or diluents may include polyols, such as dextrose, xylitol, lactitol, isomalt, mannitol such as spray dried mannitol (e.g., Pearlitol® 100SD, Pearlitol® 200SD), sorbitol, lactose, and sucrose; starches, such as pregelatinized starch, potato or corn starch; cellulose derivatives, such as powdered cellulose, microcrystalline cellulose (e.g., Avicel®) or silicified microcrystalline cellulose; talc; magnesium carbonate, calcium carbonate, calcium dihydrogen phosphate dihydrate, calcium phosphate-dibasic, calcium phosphate-tribasic, or calcium sulfate; dextrates; kaolin; silicic acid; mixtures of isomaltulose derivatives such as galenIQ™ 720; and combinations thereof. In some embodiments, the filler or diluent is mannitol.

In some embodiments, the at least one pharmaceutically acceptable filler or diluent is present an amount from about 55% to about 75% by weight (w/w). In some embodiments, the at least one filler or diluent is present in an amount from about 60% to about 70% by weight (w/w).

In some embodiments, the granular formulation suitable for reconstitution comprises at least one excipient that functions as a pharmaceutically acceptable binder. Binders may include polyvinylpyrrolidone (e.g., povidone or copovidone); cellulose derivatives, such as cellulose, such as silicified microcrystalline cellulose, microcrystalline cellulose, methyl cellulose, hydroxypropyl cellulose (HPC-L), methylcellulose, carboxymethyl cellulose sodium, hydroxypropyl methylcellulose, hydroxymethylpropylcellulose (HPMC), ethylcellulose, hydroxyl ethyl cellulose, hydroxypropylethylcellulose, or microcrystalline cellulose; starches, such as starch paste, hydroxypropyl starch, potato starch, corn starch, and pregelatinized starch; polymethacrylates (for example Eudragit RS, RL); acacia; gelatin; glucose; tragacanth; and combinations thereof. In some embodiments, the pharmaceutically acceptable binder is pregelatinized starch.

In some embodiments, the at least one pharmaceutically acceptable binder is present in an amount from about 15% to about 20% by weight (w/w). In some embodiments, the at least one binder is present in an amount from about 20% to about 30% by weight (w/w).

In some embodiments, the granular formulation suitable for reconstitution comprises at least one pharmaceutically acceptable lubricant. Examples of pharmaceutically acceptable lubricants include, but are not limited to, colloidal anhydrous silica, croscarmellose sodium, stearic acid, magnesium stearate, glyceryl behenate, calcium stearate, zinc stearate, sodium stearyl fumarate, talc, microcrystalline wax, yellow beeswax, white beeswax, corn starch, polyethylene glycol, surfactants, sodium benzoate, hydrogenated vegetable oil, hydrogenated castor oil, mineral oil, polyoxyethylene stearates, lauryl sulphate salts, glyceryl behenate, glyceryl palmitostearate, and combinations thereof. In some embodiments, the pharmaceutically acceptable lubricant is sodium stearyl fumarate.

In some embodiments, the at least one pharmaceutically acceptable lubricant is present in the pharmaceutical composition in an amount from about 0.5% to about 2% by weight (w/w) of the pharmaceutical composition. In some embodiments, the at least one lubricant is present in an amount from about 0.5% to about 1.5% by weight (w/w).

In some embodiments, the granular formulation suitable for reconstitution comprises at least one pharmaceutically acceptable coloring or flavoring agent is present in the pharmaceutical composition. The coloring agents and flavoring agents may be selected from any FDA-approved colors or flavors for oral use.

In some embodiments, the granular formulation is prepared by a process selected from dry powder blending, wet granulation, dry granulation by compaction or slugging, spray drying, hot melt extrusion, extrusion spheronization and fluidized bed granulation. In some embodiments, the granular formulation is prepared by a process selected from dry powder blending.

In some embodiments, the granular formulation is encapsulated in a capsule. In some embodiments, the method further comprises separating the parts of the capsule such that the granular formulation can be released from the capsule and reconstituted.

In some embodiments, the granular formulation is packaged in a sachet or pouch. In some embodiments, the granular formulation is packaged in a single dose sachet or pouch. In some embodiments, the granular formulation is packaged in a foil lined single dose sachet or pouch. In some embodiments, the method further comprises opening the sachet or pouch such that the granular formulation can be released and reconstituted.

In some embodiments, the granular formulation is packaged in a dual-chamber packaging system that lets patients reconstitute the granular formulation in the packaging container. Pre-dosed solvent and drug powder are stored in separate chambers; a simple twist releases the powder into the solvent at time of dosing. See, e.g., AccuRec dual chamber system available from Bormioli Pharma.

Also provided is a granular formulation suitable for reconstitution with a pharmaceutically acceptable carrier to form a stable suspension oral dosage form comprising:
(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof, in an amount of between about 5 and about 15% w/w;
at least one pharmaceutically acceptable filler or diluent, in an amount of between about 55 and about 75% w/w;
at least one pharmaceutically acceptable binder, in an amount of between about 15 and about 30% w/w; and
at least one pharmaceutically acceptable lubricant, in an amount of btween between about 0.5 and about 2% w/w.

Also provided is a granular formulation suitable for reconstitution with a pharmaceutically acceptable carrier to form a stable suspension oral dosage form comprising:
(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof, in an amount of about 10% w/w;
at least one pharmaceutically acceptable filler or diluent, in an amount of between about 60 and about 70% w/w;
at least one pharmaceutically acceptable binder, in an amount of between about 20 and about 30% w/w; and
at least one pharmaceutically acceptable lubricant, in an amount of between between about 0.5 and about 1.5% w/w.

Also provided is a granular formulation suitable for reconstitution with a pharmaceutically acceptable carrier to form a stable suspension oral dosage form comprising:
(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof, in an amount of about 10% w/w;
at least one pharmaceutically acceptable filler or diluent, in an amount of about 64% w/w;
at least one pharmaceutically acceptable binder, in an amount of about 25% w/w; and
at least one pharmaceutically acceptable lubricant, in an amount of about 1% w/w.

In some embodiments, the granular formulation suitable for reconstitution comprises:

Strength (label claim)
50 mg

| Component and Quality Standard | Quantity per unit | |
|---|---|---|
| (and Grade, if applicable) | (mg) | % |
| Compound A (GMP Grade) | 50.00 | 10.00 |
| Mannitol, Pearlitol 100SD (USP, | 318.75 | 63.75 |
| Ph. Eur., JP) | | |
| Pregelatinized Starch, Starch 1500 | 125.00 | 25.00 |
| (USP-NF, Ph. Eur., JP) | | |
| Grape Flavor | 1.25 | 0.25 |
| Sodium Stearyl Fumarate (USP-NF, | 3.75 | 0.75 |
| Ph. Eur., JP) - Intragranular | | |
| Sodium Stearyl Fumarate (USP-NF, | 1.25 | 0.25 |
| Ph. Eur., JP) - Extragranular | | |

Upon reconstitution with a pharmaceutically acceptable carrier, the granular formulation is suitable for administration to pediatrics, geriatrics, patients with swallowing difficulties i.e. dysphagic patients as well as for patients with feeding tubes in place.

Accordingly, also provided is a stable suspension oral dosage form comprising the granular formulation as described herein that has been reconstituted with a pharmaceutically acceptable carrier.

In some embodiments, the pharmaceutically acceptable carrier is a pharmaceutically acceptable liquid solvating agent.

In some embodiments, the pharmaceutically acceptable carrier is chosen from water, milk, baby formula, carbonated beverage, juice, and fruit punch. In some embodiments, the pharmaceutically acceptable carrier is chosen from milk and formula.

In some embodiments, the suspension prepared from the granular formulation provides a release of Compound A bioequivalent to an immediate release tablet having the same dose of Compound A when each is administered under fasted conditions.

In some embodiments, the suspension prepared from the granular formulation provides a release of the Compound A bioequivalent to an immediate release tablet having the same dose of Compound A when each is administered under fed conditions.

The granular formulation suitable for reconstitution also is suitable for administration via enteral feeding tubes such as NG tube and G tube. The granular formulation suitable for reconstitution is also suitable for administration to a patient via enteral feeding tubes such as NG-tube or G-tube to patients who cannot safely swallow or are unable to take medication orally. The enteral feeding tube may be a NG-tube 3.5-16 French or a G-tube of 12-28 French. Successful delivery of drug products through an enteral feeding tube requires that all of the components be able to safely pass through the feeding tube and not cause tube occlusions.

The granular formulation suitable for reconstitution can be suspended in a suitable vehicle, such as water and administered via enteral feeding tube. For patients who have a feeding tube in place, the granular formulation suitable for reconstitution is administered by a method comprising: (a) providing a granular formulation suitable for reconstitution, (b) dispersing the granular formulation suitable for reconstitution in a recommended volume of liquid medium placed in a syringe, (c) attaching the syringe to the feeding tube and delivering the contents through the feeding tube into the stomach, (d) flushing the feeding tube with an additional liquid medium to clear the tube.

The granular formulation suitable for reconstitution are suitable for administration through a NG-tube or G-tube of at least 12 French. The granules do not adhere on the walls of the tube.

3. Multiparticulate Sprinkle Dosage Form

Provided is a multiparticulate sprinkle dosage form comprising a plurality of discrete units, wherein each unit comprises one or more pharmaceutically acceptable carriers and an amount of (S)-4-((1-benzylpyrrolidin-3-yl)(methyl)amino)-2-fluoro-5-methyl-N-(thiazol-4-yl)benzenesulfonamide (Compound A) or a pharmaceutically acceptable salt thereof.

The disclosure is also directed to a multiparticulate sprinkle dosage form for use in method of treating a disease or a condition associated with Na_{V}1.6 activity. The disclosure is also directed to a multiparticulate sprinkle dosage form for use in method of treating epilepsy.

In some embodiments, the multiparticulate sprinkle dosage form comprises a plurality of discrete units, wherein each unit comprises:
(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
at least one pharmaceutically acceptable filler;
at least one pharmaceutically acceptable binder; and
at least one pharmaceutically acceptable lubricant.

The multiparticulate sprinkle dosage form comprises a plurality of discrete units which can be in the form selected from pellets, beads, particles, granules, and minitablets. In some embodiments, the discrete units are in the form of granules.

In some embodiments, the discrete unit comprises at least one pharmaceutically acceptable filler. Fillers may include polyols, such as dextrose, xylitol, lactitol, isomalt, mannitol such as spray dried mannitol (e.g., Pearlitol® 100SD, Pearlitol® 200SD), sorbitol, lactose, and sucrose; starches, such as pregelatinized starch, potato or corn starch; cellulose derivatives, such as powdered cellulose, microcrystalline cellulose (e.g., Avicel®) or silicified microcrystalline cellulose; talc; magnesium carbonate, calcium carbonate, calcium dihydrogen phosphate dihydrate, calcium phosphate-dibasic, calcium phosphate-tribasic, or calcium sulfate; dextrates; kaolin; silicic acid; mixtures of isomaltulose derivatives such as galenIQ™ 720; and combinations thereof. In some embodiments, the at least one pharmaceutically acceptable filler is mannitol.

In some embodiments, the at least one pharmaceutically acceptable filler is present an amount from about 55% to about 75% by weight (w/w). In some embodiments, the at least one pharmaceutically acceptable filler is present in an amount from about 60% to about 70% by weight (w/w).

In some embodiments, the discrete unit comprises at least one pharmaceutically acceptable binder. Binders may include polyvinylpyrrolidone (e.g., povidone or copovidone); cellulose derivatives, such as silicified microcrystalline cellulose, microcrystalline cellulose, methyl cellulose, hydroxypropyl cellulose (IPC-L), methylcellulose, carboxymethyl cellulose sodium, hydroxypropyl methylcellulose, hydroxymethylpropylcellulose (HPMC), ethylcellulose, hydroxyl ethyl cellulose, hydroxypropylethylcellulose, or microcrystalline cellulose; starches, such as starch paste, hydroxypropyl starch, potato starch, corn starch, and pregelatinized starch; polymethacrylates (for example Eudragit RS, RL); acacia; gelatin; glucose; tragacanth; and combinations thereof. In some embodiments, the at least one pharmaceutically acceptable binder is pregelatinized starch.

In some embodiments, the at least one pharmaceutically acceptable binder is present in an amount from about 15% to about 20% by weight (w/w). In some embodiments, the at least one pharmaceutically acceptable binder is present in an amount from about 20% to about 130% by weight (w/w).

In some embodiments, the discrete unit comprises at least one pharmaceutically acceptable lubricant. Examples of pharmaceutically acceptable lubricants include, but are not limited to, colloidal anhydrous silica, croscarmellose sodium, stearic acid, magnesium stearate, glyceryl behenate, calcium stearate, zinc stearate, sodium stearyl fumarate, talc, microcrystalline wax, yellow beeswax, white beeswax, corn starch, polyethylene glycol, surfactants, sodium benzoate, hydrogenated vegetable oil, hydrogenated castor oil, mineral oil, polyoxyethylene stearates, lauryl sulphate salts, glyceryl behenate, glyceryl palmitostearate, and combinations thereof. In some embodiments, the at least one pharmaceutically acceptable lubricant is sodium stearyl fumarate.

In some embodiments, the at least one pharmaceutically acceptable lubricant is present in the pharmaceutical composition in an amount from about 0.5% to about 2% by weight (w/w) of the pharmaceutical composition. In some embodiments, the at least one pharmaceutically acceptable lubricant is present in an amount from about 0.5% to about 1.5% by weight (w/w).

In some embodiments, the discrete unit comprises at least one pharmaceutically acceptable coloring or flavoring agent is present in the pharmaceutical composition. The coloring agents and flavoring agents may be selected from any FDA-approved colors or flavors for oral use.

Also provided is a multiparticulate sprinkle dosage form comprising:
(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof, in an amount of between about 5 and about 15% w/w;
at least one pharmaceutically acceptable filler, in an amount of between about 55 and about 75% w/w;
at least one pharmaceutically acceptable binder, in an amount of between about 15 and about 30% w/w; and
at least one pharmaceutically acceptable lubricant, in an amount of between between about 0.5 and about 2% w/w.

Also provided is a multiparticulate sprinkle dosage form comprising:
(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof, in an amount of about 10% w/w;
at least one pharmaceutically acceptable filler, in an amount of between about 60 and about 70% w/w;
at least one pharmaceutically acceptable binder, in an amount of between about 20 and about 30% w/w; and
at least one pharmaceutically acceptable lubricant, in an amount of between between about 0.5 and about 1.5% w/w.

Also provided is a multiparticulate sprinkle dosage form comprising:
(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof, in
an amount of about 10% w/w;
at least one pharmaceutically acceptable filler, in an amount of about 64% w/w;
at least one pharmaceutically acceptable binder, in an amount of about 25% w/w; and
at least one pharmaceutically acceptable lubricant, in an amount of about 1% w/w.

In some embodiments, the multiparticulate sprinkle dosage form comprises:

| | Amount |
|---|---|
| Ingredients | (% w/w) |
| Active Ingredient | |
| Compound A | 10.00 |
| Inactive Ingredients | |
| Mannitol | 63.75 |
| Pregelatinized Starch | 25.00 |
| Flavoring Agent | 0.25 |
| Sodium Stearyl Fumarate | 1.00 |
| Total Theoretical Weight | 100.00 |
| Hard gelatin sprinkle capsule, Size 0^{c} | 1 Capsule |

In some embodiments, the multiparticulate sprinkle dosage form comprises:

| | Amounts |
|---|---|
| Ingredients | (mg per unit) |
| Active Ingredient | |
| Compound A | 2.50 |
| Inactive Ingredients | |
| Mannitol | 15.94 |
| Pregelatinized Starch | 6.25 |
| Art Grape FL SD#554 | 0.06 |
| Sodium Stearyl Fumarate | 0.25 |
| Total Theoretical Weight | 25.00 |
| Hard gelatin sprinkle capsule, Size 0^{c} | 99.0 |

In some embodiments, the multiparticulate sprinkle dosage form comprises:

| | Amounts |
|---|---|
| Ingredients | (mg per unit) |
| Active Ingredient | |
| Compound A | 5.00 |
| Inactive Ingredients | |
| Mannitol | 31.88 |
| Pregelatinized Starch | 12.50 |
| Art Grape FL SD#554 | 0.13 |
| Sodium Stearyl Fumarate | 0.50 |
| Total Theoretical Weight | 50.00 |
| Hard gelatin sprinkle capsule, Size 0^{c} | 99.0 |

In some embodiments, the multiparticulate sprinkle dosage form comprises:

| | Amounts |
|---|---|
| Ingredients | (mg per unit) |
| Active Ingredient | |
| Compound A | 10.00 |
| Inactive Ingredients | |
| Mannitol | 63.75 |
| Pregelatinized Starch | 25.00 |
| Art Grape FL SD#554 | 0.25 |
| Sodium Stearyl Fumarate | 1.00 |
| Total Theoretical Weight | 100.00 |
| Hard gelatin sprinkle capsule, Size 0^{c} | 99.0 |

In some embodiments, the multiparticulate sprinkle dosage form comprises:

| | Amounts |
|---|---|
| Ingredients | (mg per unit) |
| Active Ingredient | |
| Compound A | 20.00 |
| Inactive Ingredients | |
| Mannitol | 127.50 |
| Pregelatinized Starch | 50.00 |
| Art Grape FL SD#554 | 0.50 |
| Sodium Stearyl Fumarate | 2.00 |
| Total Theoretical Weight | 200.00 |
| Hard gelatin sprinkle capsule, Size 0^{c} | 99.0 |

The multiparticulate sprinkle dosage form is suitable for administration to pediatrics, geriatrics, patients with swallowing difficulties i.e. dysphagic patients as well as for patients with feeding tubes in place.

The multiparticulate sprinkle dosage form may be provided in a hard capsule, which can be made from gelatin, methylcellulose, starch, or calcium alginate. The hard gelatin capsule, also known as the dry-filled capsule (DFC), consists of two sections, one slipping over the other, thus completely enclosing the active ingredient.

The multiparticulate sprinkle dosage form can be placed in contact with soft food before administration by opening the capsule to release the multiparticulate sprinkle dosage form onto the soft food. The multiparticulate sprinkle dosage form when sprinkled on acidic soft food is stable for about 90 minutes, and when sprinkled on alkaline soft food having pH more than 5 is stable for about 60 minutes. In other words, the permitted hold time of the dosage form in contact with soft food before administration is at least 30 minutes, such as at least 60 minutes. The permitted hold time of the dosage form in contact with soft food before administration is about 60 minutes for alkaline soft foods and about 90 minutes for acidic soft foods. The soft foods can have a pH ranging from 3.0 to 7.0. The acidic soft food has pH less than or equal to 5 and is selected from apple sauce, mango pudding, yogurt or cheese. The alkaline soft foods has pH more than 5 and is selected from chocolate pudding, chocolate sauce or vanilla pudding.

The multiparticulate sprinkle dosage form can be placed in contact with soft food of varying pH prior to administration. In case the food has acidic pH and the multiparticulate sprinkle dosage form after placing in contact with food are not administered immediately and have a hold time before administration, the multiparticulate sprinkle dosage form are then exposed to acidic environment in-vitro for additional time period in addition to exposure to acidic environment in stomach.

The size of discrete units suitable to be sprinkled on soft food is less than 2 mm, such as less than 1.5 mm.

In some embodiments, the pharmaceutical composition is bioequivalent to an immediate release formulation of Compound A, or a pharmaceutically acceptable salt thereof, having about the same amount of Compound A, or a pharmaceutically acceptable salt thereof.

4. General

In some embodiments, the pharmaceutical compositions disclosed herein are stable during, for example, storage, distribution, and the duration of the product's shelf-life (e.g., up to two years at room temperature/ambient conditions). A stable pharmaceutical composition may, for example, exhibit less degradation of the API and/or lower amounts of degradation products. Degradation products that arise during storage of the drug substance and/or drug product are undesirable and, in extreme cases, might even be harmful to a patient being treated with such drug product. Thus, it is desirable to control the formation of degradation products, particularly potentially harmful impurities in the drug product.

Assay and degradation product determination of pharmaceutical compositions may be performed using HPLC with UV detection.

Pharmaceutical compositions may be assessed for degradation products following storage for at least two weeks, at least one month, at least two months, at least three months, at least six months, at least twelve months, at least eighteen months, or at least twenty four months. In particular, degradation products may be assessed at time intervals of one, three, six, nine, twelve, eighteen, twenty four, thirty six, and/or forty eight months. Storage conditions may be long term, intermediate, or accelerated conditions. In particular, storage conditions may be, for example, 25° C.±2° C./40% relative humidity (RH)±5% RH, 25° C.±2° C./60% RH±5% RH, 30° C.±2° C./35% RH±5% RH, 30° C.±2° C./65% RH±5% RH, 40° C.±2° C./25% RH±5% RH, 40° C.±2° C./75% RH±5% RH, 50° C.±2° C./75% RH±5% RH, 60° C.±2° C./5% RH±5% RH, 60° C.±2° C./40% RH±5% RH, 70° C.±2° C./5% RH±5% RH, 70° C.±2° C./75% RH±5% RH, and/or 80° C.±2° C./40% RH±5% RH.

One exemplary of Compound A is (S)-4-((1-benzylpyrrolidin-3-yl)(methyl)amino)-2-fluoro-N-(thiazol-4-yl)benzenesulfonamide, (Compound B), which has the following structure:

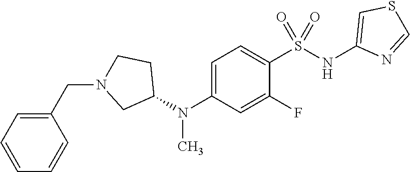 [SMILES: CN(c1ccc(S(=O)(=O)Nc2cscn2)c(F)c1)[C@H]1CCN(Cc2ccccc2)C1]

In some embodiments, Compound B is present in the granular formulation suitable for reconstitution in an amount less than about 0.5% by weight after storage for at least one month, at least two months, at least six months, at least twelve months, at least eighteen months, or at least twenty-four months at 40° C. and 75% relative humidity.

Each and every method, composition, or use described herein optionally includes the limitation “wherein the pharmaceutical composition is not a powder-in-capsule formulation of Compound A.” As used herein, a “powder-in-capsule formulation” is a formulation that does not include excipients, but rather contains only the active ingredient.

### D METHODS OF USE

Provided is a method of treating a disease or a condition associated with Na_{V}1.6 activity, the method comprising administering a pharmaceutical composition as described herein to a patient in need thereof.

Provided is a method of treating a disease or a condition associated with Na_{V}1.6 activity, the method comprising administering immediate release tablet as described herein to a patient in need thereof.

Provided is a method of administering Compound A, or a pharmaceutically acceptable salt thereof, to a patient in need thereof, comprising:
(a) providing a granular formulation as described herein;
(b) reconstituting the granular formulation to produce a stable suspension oral dosage form; and
(c) administering the stable suspension oral dosage form.

Also provided is a method of administering Compound A, or a pharmaceutically acceptable salt thereof, to a patient in need thereof, comprising: orally administering the stable suspension oral dosage form as described herein.

A method of treating a disease or a condition associated with Na_{V}1.6 activity, the method comprising administering the stable suspension oral dosage form as described herein to a patient in need thereof.

Provided is a method of administering Compound A, or a pharmaceutically acceptable salt thereof, to a patient in need thereof, comprising:
(a) providing a multiparticulate sprinkle dosage form described herein;
(b) sprinkling the discrete units on soft food; and
(c) administering the soft food orally.

In some embodiments, the soft food is chosen from applesauce, yogurt, pudding, ice cream, baby food, and a soy or grain based product.

In some embodiments, the multiparticle sprinkle dosage form is encapsulated in a capsule. In some embodiments, the method further comprises separating the parts of the capsule such that the multiparticulate sprinkle dosage form can be released from the capsule.

In some embodiments, the multiparticle sprinkle dosage form is packaged in a sachet or pouch. In some embodiments, the multiparticle sprinkle dosage form is packaged in a single dose sachet or pouch.

Also provided is a method of administering Compound A, or a pharmaceutically acceptable salt thereof, to a patient in need thereof, comprising: orally administering the multiparticulate sprinkle dosage form described herein.

Also provided is a method of administering Compound A, or a pharmaceutically acceptable salt thereof, to a patient in need thereof, comprising: orally administering a capsule containing the multiparticulate sprinkle dosage form described herein.

In some embodiments, the disease or a condition associated with Na_{V}1.6 activity is epilepsy.

In some embodiments, the disease or a condition is selected from photosensitive epilepsy, self-induced syncope, intractable epilepsy, Angelman syndrome, Dravet syndrome, benign rolandic epilepsy, CDKL5 disorder, childhood and juvenile absence epilepsy, frontal lobe epilepsy, Glut1 deficiency syndrome, hypothalamic hamartoma, infantile spasms/West's syndrome, juvenile myoclonic epilepsy, Landau-Kleffner syndrome, Lennox-Gastaut syndrome (LGS), epilepsy with myoclonic-absences, Ohtahara syndrome, Panayiotopoulos syndrome, PCDH19 epilepsy, progressive myoclonic epilepsies, Rasmussen's syndrome, ring chromosome 20 syndrome, reflex epilepsies, temporal lobe epilepsy, Lafora progressive myoclonus epilepsy, neurocutaneous syndromes, tuberous sclerosis complex, early infantile epileptic encephalopathy, early onset epileptic encephalopathy, generalized epilepsy with febrile seizures+, Rett syndrome, multiple sclerosis, Alzheimer's disease, autism, ataxia, hypotonia and paroxysmal dyskinesia.

In some embodiments, the disease or condition is epilepsy with focal onset seizures.

In some embodiments, the disease or condition is epilepsy with generalized onset seizures.

In some embodiments, the disease or condition is epilepsy with unknown onset seizures.

In some embodiments, the disease or condition is epileptic syndrome associated with mutations in SCN8A. In some further embodiments, the disease or condition is SCN8A-related developmental and epileptic encephalopathy (SCN8A-DEE).

In some embodiments, the disease or condition is Dravet syndrome.

In some embodiments, the patient is an adult patient.

In some embodiments, the patient is an adult and is administered between about 75 and 200 mg Compound A, or a pharmaceutically acceptable salt thereof, daily.

In some embodiments, the patient is an adult and is administered between about 25 and 100 mg Compound A, or a pharmaceutically acceptable salt thereof, three times daily.

In some embodiments, the patient is a pediatric patient.

In some embodiments, the patient is a pediatric patient of at least 2 years of age and is administered Compound A, or a pharmaceutically acceptable salt thereof, as shown in the table below.

| | Body Weight | Dose Level 1 | Dose Level 2 | Dose Level 3 |
|---|---|---|---|---|
| Weight Group | (kg) | (mg) TID | (mg) TID | (mg) TID |
| 1 | 10 to 20 | about 5 | about 10 | about 17.5 |
| 2 | >20 to 35 | about 10 | about 20 | about 30 |
| 3 | >35 to 50 | about 15 | about 30 | about 40 |
| 4 | >50 to 90 | about 20 | about 40 | about 60 |

TID = three times a day

In some embodiments, the patient is a pediatric patient of at least 2 years of age and is administered Compound A, or a pharmaceutically acceptable salt thereof, as shown in the table below.

| | Body Weight | Dose Level 1 | Dose Level 2 | Dose Level 3 |
|---|---|---|---|---|
| Weight Group | (kg) | (mg) BID | (mg) BID | (mg) BID |
| 1 | 10 to 20 | about 7.5 | about 15 | about 25 |
| 2 | >20 to 35 | about 15 | about 30 | about 45 |
| 3 | >35 to 50 | about 22.5 | about 45 | about 60 |
| 4 | >50 to 90 | about 30 | about 60 | about 90 |

BID = twice a day

In some embodiments, the patient is an adult patient and is administered Compound A or a pharmaceutically acceptable salt thereof in an amount of about 25 mg TID (75 mg/day), about 30 mg TID (90 mg/day), about 35 mg TID (105 mg/day), about 40 mg TID (120 mg/day), about 45 mg TID (135 mg/day), about 50 mg TID (150 mg/day), about 55 mg TID (165 mg/day), about 60 mg TID (180 mg/day), about 65 mg TID (195 mg/day), about 70 mg TID (210 mg/day), about 75 mg TID (225 mg/day), about 80 mg TID (240 mg/day), about 85 mg TID (255 mg/day), about 90 mg TID (270 mg/day), about 95 mg TID (285 mg/day), or about 100 mg TID (300 mg/day).

In some embodiments, the patient is an adult patient and is administered Compound A or a pharmaceutically acceptable salt thereof in an amount of about 30 mg BID (60 mg/day), about 35 mg BID (70 mg/day), about 40 mg BID (80 mg/day), about 45 mg BID (90 mg/day), about 50 mg BID (100 mg/day), about 55 mg BID (110 mg/day), about 60 mg BID (120 mg/day), about 65 mg BID (130 mg/day), about 70 mg BID (140 mg/day), about 75 mg BID (150 mg/day), about 80 mg BID (160 mg/day), about 85 mg BID (170 mg/day), about 90 mg BID (180 mg/day), about 95 mg BID (190 mg/day), about 100 mg BID (200 mg/day), about 110 mg BID (220 mg/day), about 120 mg BID (240 mg/day), about 130 mg BID (260 mg/day), about 140 mg BID (280 mg/day), or about 150 mg BID (300 mg/day).

### F. EXAMPLES

1. Pediatric Formulation

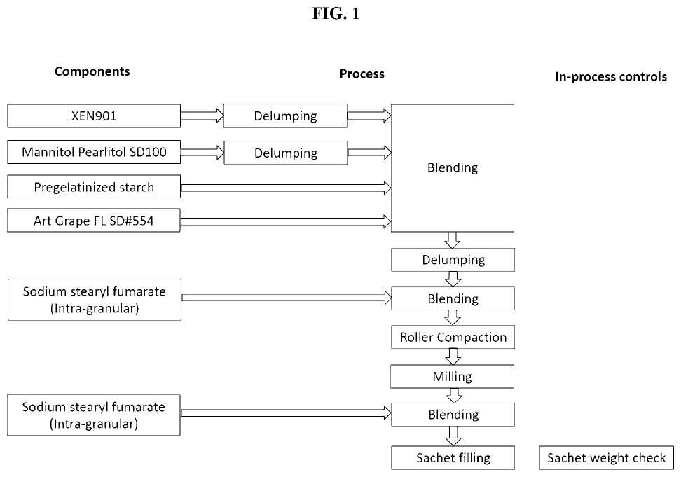

The composition of granules for oral suspension (50 mg strength) (“pediatric formulation”) comprising Compound A are presented. FIG. 1 depicts a manufacturing process for a granular formulation suitable for reconstitution as described herein. The product is packaged as a 50 mg strength dose in a foil lined sachet, for re-constitution in water.

| | | Strength (label claim) | |
|---|---|---|---|
| | | 50 mg | |
| Component and Quality Standard | | Quantity per unit | |
| (and Grade, if applicable) | Function | (mg) | % |
| Compound A (GMP Grade) | API | 50.00 | 10.00 |
| Mannitol, Pearlitol 100SD (USP, | Diluent/Filler | 318.75 | 63.75 |
| Ph. Eur., JP) | | | |
| Pregelatinized Starch, Starch 1500 | Disintegrant/Binder | 125.00 | 25.00 |
| (USP-NF, Ph. Eur., JP) | | | |
| Grape Flavor | Flavor Masker | 1.25 | 0.25 |
| Sodium Stearyl Fumarate (USP-NF, | Lubricant | 3.75 | 0.75 |
| Ph. Eur., JP) - Intragranular | | | |
| Total Intragranular Components | | 498.75 | 99.75 |
| Sodium Stearyl Fumarate (USP-NF, | Lubricant | 1.25 | 0.25 |
| Ph. Eur., JP) - Extragranular | | | |
| Total | | 500.00 | 100.00 |

Stability data has been collected on a batch of 50 mg granules, having the above components. The results indicate that the granules are stable for at least two months (8 weeks) when stored at accelerated storage conditions (40° C./75% RH).

2. Immediate Release Tablet

FIG. 1 depicts a manufacturing process for the immediate release tablets as described herein and having the following composition:

| | Strength (label claim) |
|---|---|
| | 50 mg |
| | Batch Size(s) (number of dosage units) |
| Component and Quality Standard | 1,600 tablets (theoretical batch size) |
| (and Grade, if applicable) | Quantity per batch (g) |
| XEN901 API (GMP Grade) | 80.00 |
| Microcrystalline Cellulose, Pharmacel 102 (USP-NF, Ph. Eur.) | 72.80 |
| Croscarmellose Sodium, Primellose (USP-NF, Ph. Eur., JP) | 4.80 |
| Silica Colloidal Anhydrous, Aerosil (USP/NF, Ph. Eur., JP) | 0.80 |
| Sodium Lauryl Sulfate, Kolliphor SLS (NF, Ph. Eur., BP, JP) | 0.80 |
| Magnesium Stearate (NF, Ph. Eur.) | 0.80 |
| Core Total | 160.00 |
| Coating | |
| Opadry II (white) | 10.40 |
| Water for injection, (USP-NF, Ph. Eur.) | 104.00* |
| Final Total | 170.40 |

Stability data has been collected on a Compound A 50 mg IR tablets, manufactured with the formulation above. The results obtained to date indicate that the Compound A 50 mg IR tablets are stable for at least two months when stored at accelerated storage conditions (40° C./75% RH).

3. Drug Product

The composition of granules for oral suspension (50 mg strength) (“pediatric formulation”) comprising Compound A are presented. FIG. 1 depicts a manufacturing process for a granular formulation suitable for reconstitution as described herein. The product is packaged as a 50 mg strength dose in a foil lined sachet, for re-constitution in water.

| | | Strength (label claim) | |
|---|---|---|---|
| | | 50 mg | |
| Component and Quality Standard | | Quantity per unit | |
| (and Grade, if applicable) | Function | (mg) | % |
| Compound A (GMP Grade) | API | 50.00 | 10.00 |
| Mannitol, Pearlitol 100SD (USP, | Diluent/Filler | 318.75 | 63.75 |
| Ph. Eur., JP) | | | |
| Pregelatinized Starch, Starch 1500 | Disintegrant/Binder | 125.00 | 25.00 |
| (USP-NF, Ph. Eur., JP) | | | |
| Grape Flavor | Flavor Masker | 1.25 | 0.25 |
| Sodium Stearyl Fumarate (USP-NF, | Lubricant | 3.75 | 0.75 |
| Ph. Eur., JP) - Intragranular | | | |
| Total Intragranular Components | | 498.75 | 99.75 |
| Sodium Stearyl Fumarate (USP-NF, | Lubricant | 1.25 | 0.25 |
| Ph. Eur., JP) - Extragranular | | | |
| Total | | 500.00 | 100.00 |

Stability data has been collected on a batch of 50 mg granules, having the above components. The results indicate that the granules are stable for at least two months (8 weeks) when stored at accelerated storage conditions (40° C./75% RH).

4. Drug Product

The drug product is a hard gelatin capsule containing oral granules for sprinkle. The oral granules are contained and manually released from the sprinkle capsule by the integral twist-locking mechanism that requires a simple quarter turn to open, upon which the granules can be sprinkled.

The formulation and manufacture of oral granules for sprinkle capsule 2.5 mg, 5 mg, 10 mg, and 20 mg follow a multi-step manufacturing process which can be divided into two major steps: Step 1, the formulation and manufacture of a roller compacted common blend (drug product intermediate) and Step 2, encapsulation of the common blend intermediate into Size 0 Hard Gelatin Sprinkle Capsules.

Step 1 is initiated by de-agglomeration of Compound A drug substance and mannitol. These two components, along with pregelatinized starch and artificial grape flavor, are then added to a V-shell blender and mixed for 5 minutes at 24 rpm. This blend is de-agglomerated, added back to the blender, and mixed with sodium stearyl fumarate for 5 minutes at 24 rpm to form the intra-granular blend.

The intra-granular blend is roller compacted and milled. The roller compacted and milled material is blended with an extra-granular portion of sodium stearyl fumarate. This concludes preparation of the drug product intermediate.

The intermediate common blend is filled into Size 0 hard gelatin sprinkle capsules using an Xcelodose®, Profiller®, or manual fill. The capsules are filled to the target fill weight to achieve the indicated dose (2.5 mg, 5 mg, 10 mg, and 20 mg of active ingredient).

The composition of oral granules for sprinkle capsule comprising 2.5, 5, 10, and 20 mg of Compound A are presented below.

Unit Composition, Pharmaceutical Function, and Ingredients

| | Common | | | | | |
|---|---|---|---|---|---|---|
| | Blend | 2.5 mg | 5 mg | 10 mg | 20 mg | Pharmaceutical |
| Ingredients | (% w/w) | (mg per unit) | (mg per unit) | (mg per unit) | (mg per unit) | Function |
| Active | | | | | | |
| Ingredient | | | | | | |
| Compound A | 10.00 | 2.50 | 5.00 | 10.00 | 20.00 | Active |
| Inactive | | | | | | |
| Ingredients | | | | | | |
| Mannitol | 63.75 | 15.94 | 31.88 | 63.75 | 127.50 | Filler |
| Pregelatinized | 25.00 | 6.25 | 12.50 | 25.00 | 50.00 | Binder/ |
| Starch | | | | | | Disintegrant |
| Art Grape | 0.25 | 0.06 | 0.13 | 0.25 | 0.50 | Flavor |
| FL SD# 554 | | | | | | |
| Sodium | 1.00 | 0.25 | 0.50 | 1.00 | 2.00 | Lubricant |
| Stearyl | | | | | | |
| Fumarate | | | | | | |
| Total | 100.00 | 25.00 | 50.00 | 100.00 | 200.00 | NA |
| Theoretical | | | | | | |
| Weight | | | | | | |
| Hard | 1 | 99.0 | 99.0 | 99.0 | 99.0 | Capsule |
| gelatin | Capsule | | | | | Shell, |
| sprinkle | | | | | | Easy Open |
| capsule, | | | | | | Sprinkle |
| Size 0^{c} | | | | | | Design |

5. Stability Data

Stability data for oral granules for sprinkle capsule comprising 2 and 30 mg of Compound A are presented in the tables below.

Stability Data for Oral Granules for Sprinkle Capsule, 2 mg Stored at 2-8° C.

| | | | Time Points (Month) | |
|---|---|---|---|---|
| Test | Specifications | | Initial | 1 |
| Description | White opaque size 0 hard | | White opaque size 0 | White opaque size 0 |
| | shell capsule containing white | | hard shell capsule | hard shell capsule |
| | to light brown granules. | | containing white | containing white |
| | | | granules. | granules. |
| Assay (% label | 90.0% to 110.0% | | 99.6% | 98.8% |
| claim) | | | | |
| Degradation | Compound B NMT 0.5% | | 0.23% | 0.21% |
| Products (% | | | | |
| area) | | | | |
| | Any unspecified | RRT | 0.08% | 0.08% |
| | degradation product | 0.79 | 0.06% | 0.06% |
| | NMT 0.2% | RRT | | |
| | | 1.18 | | |
| | | RRT | 0.08% | 0.06% |
| | | 1.28 | | |

| | | | |
|---|---|---|---|
| | Total Degradation Products | 0.5% | 0.4% |
| | NMT 3.0% | | |
| Dissolution | NLT 85% (Q = 80%) in | 96% | 96% |
| | 30 minutes | | |

Stability Data for Oral Granules for Sprinkle
Capsule, 2 mg Stored at 25° C./60% RH

| | | | Time Points (Month) | | |
|---|---|---|---|---|---|
| Test | Specifications | | Initial | 1 | 3 |
| Description | White opaque size 0 | | White opaque | White opaque | White opaque |
| | hard shell capsule | | size 0 hard | size 0 hard | size 0 hard |
| | containing white to light | | shell capsule | shell capsule | shell capsule |
| | brown granules. | | containing | containing | containing |
| | | | white granules. | white granules. | white granules. |
| Assay (% | 90.0% to 110.0% | | 99.6% | 101.1% | 98.8% |
| label claim) | | | | | |
| Degradation | Compound B | | 0.23% | 0.21% | 0.23% |
| Products (% | NMT 0.5% | | | | |
| area) | | | | | |
| | Any | RRT | 0.08% | 0.08% | 0.08% |
| | unspecified | 0.79 | 0.06% | 0.06% | 0.06% |
| | degradation | RRT | 0.08% | 0.06% | 0.09% |
| | product | 1.18 | | | |
| | NMT 0.2% | RRT | | | |
| | | 1.27 | | | |

| | | | | |
|---|---|---|---|---|
| | Total Degradation | 0.5% | 0.4% | 0.5% |
| | Products | | | |
| | NMT 3.0% | | | |
| Dissolution | NLT 85% (Q = 80%) in | 96% | 98% | 93% |
| | 30 minutes | | | |

Stability Data for Oral Granules for Sprinkle
Capsule, 2 mg Stored at 40° C./75% RH

| | | | Time Points (Month) | | |
|---|---|---|---|---|---|
| Test | Specifications | | Initial | 1 | 3 |
| Description | White opaque size 0 | | White opaque | White opaque | White opaque |
| | hard shell capsule | | size 0 hard | size 0 hard | size 0 hard |
| | containing white to light | | shell capsule | shell capsule | shell capsule |
| | brown granules. | | containing | containing | containing |
| | | | white granules. | white granules. | white granules. |
| Assay (% | 90.0% to 110.0% | | 99.6% | 99.5% | 98.7% |
| label claim) | | | | | |
| Degradation | Compound B | | 0.23% | 0.22% | 0.23% |
| | NMT 0.5% | | | | |
| Products (% | Any | RRT | 0.08% | 0.08% | 0.08% |
| area) | unspecified | 0.79 | 0.06% | 0.06% | 0.06% |
| | degradation | RRT | 0.08% | 0.09% | 0.11% |
| | product | 1.18 | | | |
| | NMT 0.2% | RRT | | | |
| | | 1.28 | | | |

| | | | | |
|---|---|---|---|---|
| | Total Degradation | 0.5% | 0.5% | 0.5% |
| | Products NMT 3.0% | | | |
| Dissolution | NLT 85% (Q = 80%) in | 96% | 101% | 99% |
| | 30 minutes | | | |

Stability Data for Oral Granules for Sprinkle Capsule, 30 mg Stored at 2-8° C.

| | | | Time Points (Month) | |
|---|---|---|---|---|
| Test | Specifications | | Initial | 1 |
| Description | White opaque size 0 hard | | White opaque size 0 | White opaque size 0 |
| | shell capsule containing white | | hard shell capsule | hard shell capsule |
| | to light brown granules. | | containing white | containing white |
| | | | granules. | granules. |
| Assay (% label | 90.0% to 110.0% | | 100.2% | 100.6% |
| claim) | | | | |
| Degradation | Compound B | | 0.22% | 0.21% |
| Products (% | NMT 0.5% | | | |
| area) | | | | |
| | Any unspecified | RRT | 0.07% | 0.08% |
| | degradation product | 0.79 | | |
| | NMT 0.2% | RRT | 0.06% | 0.06% |
| | | 1.18 | | |
| | | RRT | 0.07% | 0.06% |
| | | 1.27 | | |

| | | | |
|---|---|---|---|
| | Total Degradation Products | 0.4% | 0.4% |
| | NMT 3.0% | | |
| Dissolution | NLT 85% (Q-80%) in | 98% | 98% |
| | 30 minutes | | |

Stability Data for Oral Granules for Sprinkle
Capsule, 30 mg Stored at 25° C./60% RH

| | | | Time Points (Month) | | |
|---|---|---|---|---|---|
| Test | Specifications | | Initial | 1 | 3 |
| Description | White opaque size 0 | | White opaque | White opaque | White opaque |
| | hard shell capsule | | size 0 hard | size 0 hard | size 0 hard |
| | containing white to light | | shell capsule | shell capsule | shell capsule |
| | brown granules. | | containing | containing | containing |
| | | | white granules. | white granules | white granules |
| Assay (% | 90.0% to 110.0% | | 100.2% | 100.2% | 99.9% |
| label claim) | | | | | |
| Degradation | Compound B | | 0.22% | 0.21% | 0.22% |
| | NMT 0.5% | | | | |
| Products (% | Any | RRT | 0.07% | 0.08% | 0.08% |
| area) | unspecified | 0.79 | 0.06% | 0.06% | 0.06% |
| | degradation | RRT | 0.07% | 0.06% | 0.08% |
| | product | 1.18 | | | |
| | NMT 0.2% | RRT | | | |
| | | 1.28 | | | |

| | | | | |
|---|---|---|---|---|
| | Total Degradation | 0.4% | 0.4% | 0.4% |
| | Products NMT 3.0% | | | |
| Dissolution | NLT 85% (Q = 80%) in | 98% | 97% | 99% |
| | 30 minutes | | | |

Stability Data for Oral Granules for Sprinkle
Capsule, 30 mg Stored at 40° C./75% RH

| | | | Time Points (Month) | | |
|---|---|---|---|---|---|
| Test | Specifications | | Initial | 1 | 3 |
| Description | White opaque size 0 | | White opaque | White opaque | White opaque |
| | hard shell capsule | | size 0 hard | size 0 hard | size 0 hard |
| | containing white to light | | shell capsule | shell capsule | shell capsule |
| | brown granules. | | containing | containing | containing |
| | | | white granules. | white granules. | white granules. |
| Assay (% | 90.0-110.0% | | 100.2% | 100.1% | 101.4% |
| label claim) | | | | | |
| Degradation | Compound B NMT | | 0.22% | 0.22% | 0.23% |
| | 0.5% | | | | |
| Products (% | Any | RRT | 0.07% | 0.08% | 0.08% |
| area) | unspecified | 0.79 | 0.06% | 0.06% | 0.06% |
| | degradation | RRT | 0.07% | 0.08% | 0.11% |
| | product | 1.18 | | | |
| | NMT 0.2% | RRT | | | |
| | | 1.28 | | | |

| | | | | |
|---|---|---|---|---|
| | Total Degradation | 0.4% | 0.4% | 0.5% |
| | Products NMT 3.0% | | | |
| Dissolution | NLT 85% (Q = 80%) in | 98% | 99% | 100% |
| | 30 minutes | | | |

6. Pharmacokinetic Study

In this single center, open-label, randomized study, 24 subjects (8 per each arm) received Compound A adult immediate release tablet or granular formulation suitable for reconstitution formulation in a fasted or fed state (high fat meal) in a 3-period, 3-sequence crossover design. Compound A was formulated as a granular formulation suitable for reconstitution (“Compound A pediatric formulation”) as described herein.

Patient demographics are as shown below:

Gender, n (%)

| | | |
|---|---|---|
| Male | 16 | (66.7) |
| Female | 8 | (33.3) |

Age at informed consent (years)

| | |
|---|---|
| Mean | 37.0 |
| SD | 10.3 |
| Range | 20-54 |

Race, n (%)

| | | |
|---|---|---|
| Caucasian | 15 | (62.5) |
| Black | 5 | (20.8) |
| Asian | 2 | (8.3) |
| Other | 2 | (8.3) |

Ethnicity

| | | |
|---|---|---|
| Not Hispanic or Latino | 23 | (95.8) |
| Hispanic or Latino | 1 | (4.2) |

BMI (kg/m²)

| | |
|---|---|
| Mean | 25.4 |
| SD | 2.7 |

A 50 mg dose was used in this adult PK study.

For each study period, subjects received one of the 3 following treatments:

Treatment A: Single doses of 50 mg Compound A pediatric formulation (1 sachet of granules) under fed conditions.

Treatment B: Single doses of 50 mg Compound A pediatric formulation (1 sachet of granules) under fasting conditions.

Treatment C: Single doses of 50 mg Compound A adult tablet formulation (1×50 mg IR tablet) under fasting conditions.

Three groups of 8 subjects were randomized to one of the following 3 sequences.

| | Period 1 | Period 2 | Period 3 |
|---|---|---|---|
| Sequence ABC | Treatment A | Treatment B | Treatment C |
| N = 8 | | | |
| Sequence CAB | Treatment C | Treatment A | Treatment B |
| N = 8 | | | |
| Sequence BCA | Treatment B | Treatment C | Treatment A |
| N = 8 | | | |

N: number of subjects.
Treatment A = Pediatric Formulation, 50 mg, Fed; Treatment B = Pediatric Formulation, 50 mg, Fasting; Treatment C = Adult Tablet Formulation, 50 mg, Fasting.

### Treatment A: Compound A Pediatric Formulation (Fed Conditions)

Following an overnight fast of at least 10 hours, subjects received a standardized high-fat, high calorie meal 30 minutes before administration. Subjects must have eaten the total content of this meal in 30 minutes or less.

Thirty minutes after the start of breakfast, subjects were administered the suspension (the contents of one sachet of the Compound A pediatric formulation [granules for oral suspension] was mixed thoroughly with 120 mL of water at ambient temperature). Thereafter, the glass was rinsed twice with approximately 60 mL of water at ambient temperature and the subject was asked to drink the 60 mL of suspension each time. A total of approximately 240 mL of water was administered with the formulation within 5 minutes.

Additional food was then withheld for at least 4 hours following administration. Water was provided as needed until 1 hour pre-dose. Water was allowed beginning 1 hour after the administration of the drug.

### Treatment B: Compound a Pediatric Formulation (Fasted Conditions)

Subjects fasted overnight for a minimum of 10 hours prior to dosing.

Subjects were administered the Compound A suspension (the contents of one sachet [granules for oral suspension] was mixed thoroughly with 120 mL of water at ambient temperature). Thereafter, the glass was rinsed twice with approximately 60 mL of water at ambient temperature and the subject was asked to drink the 60 mL of suspension each time. A total of approximately 240 mL of water was administered with the formulation within 5 minutes.

Fasting continued for at least 4 hours following Compound A administration. Water was provided as needed until 1 hour pre-dose. Water was allowed beginning 1 hour after the administration of the drug.

### Treatment C: Compound a IR Tablet

Subjects fasted overnight for a minimum of 10 hours prior to dosing.

A single dose of the Compound A IR tablet was administered with approximately 240 mL of water, starting at 09:00, to 1 subject per 2 minute intervals.

Fasting continued for at least 4 hours following administration. Water was provided as needed until 1 hour pre-dose. Water was allowed beginning 1 hour after the administration of the drug.

### Pharmacokinetic Results

Serial PK samples were obtained pre-dose and at specific timepoints through 48 h post-dose Plasma concentrations of Compound A were determined using validated liquid chromatography with tandem mass spectrometry method.

Following the 50 mg single-dose administration of Compound A adult tablet formulation compared to the 50 mg single-dose administration of Compound A pediatric formulation under fasting conditions, Compound A in both formulations rapidly appeared in plasma with median tₘₐₓ of approximately 1 h.

Compound A Cₘₐₓ, AUC₀₋ₜ, and AUC_{0-inf} were comparable between treatments with mean values of 2239 ng/mL, 12109 ng*h/mL, and 12381 ng*h/mL for the adult tablet formulation and 2023 ng/mL, 11094 ng*h/mL, and 11304 ng*h/mL for the pediatric formulation, respectively. The GMR (with associated 90% CI) were 91.33% (81.48%-102.38%), 93.21 (88.02%-98.70%), and 92.84% (88.03%-97.92%) for Cₘₐₓ, AUC₀₋ₜ and AUC_{0-inf}, respectively. When comparing the pediatric formulation to the adult tablet formulation under fasting conditions, the intra-subject CV % was 11% for the AUCs and 23% for Cₘₐₓ.

The elimination half-life, apparent volume of distribution, and apparent total plasma clearance were comparable between formulations (8.5 h for T_{1/2}, 55 L for V_{Z}/F, and 5 L/h for CL/F).

Following the 50 mg single-dose administration of Compound A pediatric formulation in a fed state (high fat meal) compared to its single-dose administration in a fasted state, the time to maximum plasma concentrations was delayed by 2 hours after the intake of the pediatric formulation with a high-fat meal.

Compound A total systemic exposure, as indicated by the AUC₀₋ₜ and AUC_{0-inf}, was comparable between the fasted and the fed state with respective mean values of 11094 ng*h/mL and 11304 ng*h/mL in a fasting conditions and 11667 ng*h/mL and 11770 ng*h/mL in fed conditions. A GMR (with associated 90% CI) of 103.74% (98.23-109.57%) and 102.67% (97.98-107.58%) were observed for AUC₀₋ₜ and AUC_{0-inf}, respectively. The intra-subject CV % were 11% and 9%, respectively.

The administration of Compound A pediatric formulation with a high-fat meal decreased the Cₘₐₓ by approximately 38%, the GMR (90% CI) was 61.63% (54.84%-69.26%). The mean Cₘₐₓ values were 2023 ng/mL and 1247 ng/mL, respectively. The intra-subject CV % was 24%.

The elimination half-life were 8 h and 6 h, the apparent volume of distribution were 57 L and 42 L and apparent total plasma clearance was 4.6 L/h under fasted and fed states, respectively.

Following the single-dose administration of Compound A adult 50 mg tablet formulation compared to the single-dose administration of Compound A 50 mg Pediatric Formulation under fasting conditions, the mean amount and percentage of the Compound A dose administered that was excreted unchanged in urine over a period of 48 hours following dosing were 30656 ng and 0.06% for the adult tablet formulation and 34424 ng and 0.07% for the pediatric formulation.

Compound A CLr was 2.4 mL/h for the Adult Tablet Formulation and 3.1 mL/h for the Pediatric Formulation (GMR: 120.89%).

Urine pre-dose concentrations were observed in Periods 2 and/or 3 in 14 out of the 24 subjects in Cohort 2. These pre-dose concentrations ranged from 0.102 ng/mL to 3.627 ng/mL, with the highest pre-dose concentrations observed for Subjects 212 and 216. Overall (based on data from Cohorts 1 and 2), the fraction excreted in urine is less than 0.1%, thus the contribution of the urinary excretion in the disposition and elimination of the Compound A as a parent compound is negligible. The PK and statistical analysis were performed including all subjects with measurable pre-dose concentrations.

Following the single-dose 50 mg administration of Compound A pediatric formulation in a fed state (high fat meal) compared to its single-dose administration in a fasted state, the mean amount and percentage of the Compound A dose administered that was excreted unchanged in urine over a period of 48 hours following dosing were 34424 ng and 0.07% under fasting condition and 41205 ng and 0.08% under fed condition.

Compound A CLr was 3 mL/h under fasting condition and 3.3 mL/h under fed condition (GMR: 115.56%).

Administration of a single 50 mg dose of Compound A pediatric formulation (1 sachet) under fed state (ie, with a high-fat meal) delayed the rate, but not the extent of Compound A absorption/total exposure, compared to that of the fasted state. A single 50 mg dose of Compound A pediatric formulation (1 sachet) is bioequivalent to a single 50 mg dose of Compound A adult tablet formulation (1×50 mg IR tablet) under fasted conditions. Compound A was only negligibly excreted in urine as unchanged parent (fe of 0.06 to 0.08%).

The pharmaceutical compositions, methods, and uses described herein will be better understood by reference to the following exemplary embodiments and examples, which are included as an illustration of and not a limitation upon the scope of the invention.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations, or methods, or any combination of such changes and modifications of use of the invention, may be made without departing from the spirit and scope thereof.

All references (patent and non-patent) cited above are incorporated by reference into this patent application. The discussion of those references is intended merely to summarize the assertions made by their authors. No admission is made that any reference (or a portion of any reference) is relevant prior art (or prior art at all). Applicant reserves the right to challenge the accuracy and pertinence of the cited references.

1. An immediate release tablet comprising:
(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
at least one pharmaceutically acceptable diluent;
at least one pharmaceutically acceptable disintegrant;
at least one pharmaceutically acceptable glidant;
at least one pharmaceutically acceptable wetting agent; and
at least one pharmaceutically acceptable lubricant.

2. The immediate release tablet of claim 1, further comprising a film coating.

3. The immediate release tablet of claim 2, wherein the film coating is an aqueous film.

4. The immediate release tablet of claim 3, wherein the aqueous film coating comprises Opadry II.

5. A granular formulation suitable for reconstitution with a pharmaceutically acceptable carrier to form a stable suspension oral dosage form comprising one or more pharmaceutically acceptable excipients and an amount of (S)-4-((1-benzylpyrrolidin-3-yl)(methyl)amino)-2-fluoro-5-methyl-N-(thiazol-4-yl)benzenesulfonamide (Compound A) or a pharmaceutically acceptable salt thereof.

6. The granular formulation of claim 5, wherein the one or more pharmaceutically acceptable excipients comprises at least one pharmaceutically acceptable filler or diluent.

7. The granular formulation of claim 5, wherein the one or more pharmaceutically acceptable excipients comprises at least one pharmaceutically acceptable binder.

8. The granular formulation of claim 5, wherein the one or more pharmaceutically acceptable excipients comprises at least one pharmaceutically acceptable lubricant.

9. The granular formulation of claim 5, wherein the granular formulation is prepared by a process selected from dry powder blending, wet granulation, dry granulation by compaction or slugging, spray drying, hot melt extrusion, extrusion spheronization and fluidized bed granulation.

10. A granular formulation suitable for reconstitution with a pharmaceutically acceptable carrier to form a stable suspension oral dosage form comprising:
(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
at least one pharmaceutically acceptable filler or diluent,
at least one pharmaceutically acceptable binder, and
at least one pharmaceutically acceptable lubricant.

11. A stable suspension oral dosage form comprising the granular formulation of claim 5, that has been reconstituted with a pharmaceutically acceptable carrier.

12. The stable suspension oral dosage form of claim 11, wherein the pharmaceutically acceptable carrier is a pharmaceutically acceptable liquid.

13. The stable suspension oral dosage form of claim 11, wherein the pharmaceutically acceptable carrier is chosen from water, milk, baby formula, carbonated beverage, juice, and fruit punch.

14. A method of administering Compound A, or a pharmaceutically acceptable salt thereof, to a patient in need thereof, comprising:
(a) providing a granular formulation of claim 5;
(b) reconstituting the granular formulation to produce a stable suspension oral dosage form; and
(c) administering the stable suspension oral dosage form.

15. A method of administering Compound A, or a pharmaceutically acceptable salt thereof, to a patient in need thereof, comprising: orally administering the stable suspension oral dosage form of claim 11.

16. A multiparticulate sprinkle dosage form comprising a plurality of discrete units, wherein each unit comprises one or more pharmaceutically acceptable carriers and an amount of (S)-4-((1-benzylpyrrolidin-3-yl)(methyl)amino)-2-fluoro-5-methyl-N-(thiazol-4-yl)benzenesulfonamide (Compound A) or a pharmaceutically acceptable salt thereof.

17. The multiparticulate sprinkle dosage form of claim 16, wherein the discrete unit further comprises at least one pharmaceutically acceptable filler.

18. The multiparticulate sprinkle dosage form of claim 16, wherein the discrete unit further comprises at least one pharmaceutically acceptable binder.

19. The multiparticulate sprinkle dosage form of claim 16, wherein the discrete unit further comprises at least one pharmaceutically acceptable lubricant.

20. A multiparticulate sprinkle dosage form comprising a plurality of discrete units, wherein each unit comprises:
(S)-4-((1-benzylpyrrolidin-3-yl)(methyl)amino)-2-fluoro-5-methyl-N-(thiazol-4-yl)benzenesulfonamide (Compound A), or a pharmaceutically acceptable salt thereof,
at least one pharmaceutically acceptable filler,
at least one pharmaceutically acceptable binder, and
at least one pharmaceutically acceptable lubricant.

21. A method of administering Compound A, or a pharmaceutically acceptable salt thereof, to a patient in need thereof, comprising:
(a) providing a multiparticulate sprinkle dosage form of claim 16;
(b) sprinkling the discrete units on soft food, and
(c) administering the soft food orally.

22. The method of claim 21, wherein the soft food is chosen from applesauce, yogurt, pudding, ice cream, baby food, and a soy or grain based product.

23. The immediate release tablet of claim 1, wherein Compound A is present in an amount of between about 5 and about 15% w/w, measured as the free base.

24. A method of treating a disease or a condition associated with Na_{V}1.6 activity in a patient in need thereof, the method comprising: administering to the patient in need thereof a therapeutically effective amount of an immediate release tablet of claim 1.

25. The method of claim 24, wherein disease or a condition associated with Na_{V}1.6 activity is epilepsy.
